(19) United States
(12) Patent Application Publication
(10) Pub. No.: US 2023/0018532 A1
(43) Pub. Date: Jan. 19, 2023

(54) MOTORIZED ACTUATION MODULE FOR ACTUATING AN ENDOSCOPIC INSTRUMENT

(71) Applicant: INSTITUT HOSPITALO-UNIVERSITAIRE DE CHIRURGIE MINI-INVASIVE GUIDEE PAR L'IMAGE, Strasbourg Cedex (FR)

(72) Inventors: Albert SERRA-TORRENT, BARCELONA (ES); Andras LEGNER, Wien (AT); Eugene SKELTON, Dublin (IE); Anthony WRIGHT, Dunqarvan (IE)

(73) Assignee: INSTITUT HOSPITALO-UNIVERSITAIRE DE CHIRURGIE MINI-INVASIVE GUIDEE PAR L'IMAGE, Strasbourg Cedex (FR)

(21) Appl. No.: 17/781,903

(22) PCT Filed: Dec. 2, 2020
(86) PCT No.: PCT/FR2020/052253

§ 371 Date: Jun. 2, 2022

(30) Foreign Application Priority Data
Dec. 3, 2019 (FR) 1913678

(51) Int. Cl. A61B 1/005; A61B 1/00; A61B 1/015; A61B 1/018

(52) U.S. Cl. CPC A61B 1/0052; A61B 1/00133; A61B 1/00006; A61B 1/015; A61B 1/018; A61B 1/126

(57) ABSTRACT

A motorized actuation module actuates an endoscopic instrument. It is made up of a mounting plate able to be attached to the ulnar-palmar grasping zone of a handgrip of an endoscope, the mounting plate having an ulnar-palmar bearing surface extended by a heel extending in a direction that makes an angle of 90°±25° with respect to the perpendicular to the plane of the ulnar-palmar bearing surface. The heel includes an electromechanical sensor that delivers a control signal controlling the movement of an endoscopy instrument.

### CROSS-REFERENCE TO RELATED APPLICATIONS

This application claims priority to PCT Patent Application Serial No. PCT/FR2020/052253 filed on Dec. 2, 2020, which claims priority to French Patent Application Serial No. 1913678 filed Dec. 3, 2019, both of which are incorporated by reference herein.

### TECHNICAL FIELD

The present invention relates to the field of flexible endoscopy, more particularly a system for controlling the translational movement of flexible endoscopic tools in an outgoing flexible endoscope. A flexible endoscope comprises a handgrip with control buttons that allow the flexible end of the endoscope rod to be moved in different directions. The user holds the handgrip with the left hand, the fingers of which can operate the various adjustment buttons, while the right hand guides the insertion area of the flexible rod.

Initially, flexible endoscopes were mainly used for diagnosis and in particular for in vivo imaging of hollow bodies. However, the therapeutic uses of flexible endoscopes have developed over recent decades, implementing additional micro-surgical instruments inserted through a working channel present on the endoscope or attached to the outside of the endoscope. These accessory instruments generally have at least one mechanism for actuating and/or moving and positioning the active distal head.

During therapeutic use of a flexible endoscope, the user must actuate and control the insertion, that is to say, the advancement and the retraction, of the distal instrument, generally with the right hand. As the movement is transmitted along the endoscopic rod, the insertion area of which is no longer guided by the user's hand, the position of the camera changes and causes the target to be lost from view. The coordination of these actions often disrupts the continuity of the workflow and results in a longer procedural time. To solve this problem, a second operator/assistant is often needed to hold the endoscope rod, while the main operator uses his free hand to manually control the flexible tool on the flexible endoscope handgrip.

This requires learning cycles to achieve good communication and coordination between the operator and the assistant, without completely avoiding work disruptions, as it is often difficult to specify the angle, the location, the moment and a sequence of desired movements. Therefore, various automated tool control devices for flexible endoscopes have been proposed to overcome these difficulties.

### BACKGROUND

To facilitate the control of the movement of the instrument without using an assistant, solutions have been proposed in the state of the art where the movement is motorized and where the handgrip has a movement control. Known in the state of the art is international patent application WO2015/029041 describing a palm interface that can be engaged by the palm of a hand, a restraint capable of deforming elastically to apply a restraining force on the back of the hand and a finger interface engageable by one or more fingers of said hand.

The solution proposed by this document comprises:
a) An interface mounted on a pivotal support attached to a housing of the control unit, the first interface being engageable by the palm of a hand;
b) a restraint pivotally attached to the first interface and having an element capable of elastically deforming to apply a restraining force to the back of the hand when the palm is engaged with the first interface; and
c) a second interface pivotally attached to the first interface and engageable by one or more fingers of the hand.

Also known is European patent EP2106735 describing an endoscope configured to be carried with one hand, comprising:
an insertion portion extending in an axial longitudinal direction, comprising a distal end portion and a proximal end portion and configured to be inserted into a body; and
an operation portion coaxially connected to the proximal end portion of the insertion portion, extending in the axial longitudinal direction and configured to be held and operated by an operator, and
wherein the insertion portion comprises a bending portion configured to bend.

The operation portion comprises:
a first grasping portion extending in the axial longitudinal direction and configured to be grasped by an operator;
a bending operation portion body arranged on a proximal end end side in the axial longitudinal direction as the first grasping portion and comprising a proximal end portion;
extends widthwise and is configured to move along the axial longitudinal direction by rotation of the support portion, wherein at least one of an operator's thumb and fingers can be placed on the actuation finger placement portion; and
a radio unit connected to the proximal end portion of the bending operation portion body and configured to perform radio communication.

A first drawback of the solutions of the prior art relates to the ergonomics of the control members and their configuration, which is not compatible with the handgrips of standard flexible endoscopes. They therefore involve significant learning time for the practitioner. Some solutions require users to combine the thumb and the middle or ring finger to manipulate their knobs, which prevents simultaneous actuation of the suction or wash valves. A second drawback lies in the fact that these solutions require the complete redesign of a handgrip, and the replacement of existing endoscopes with new endoscopes.

International patent application WO2017025969 describes a control unit that can be attached to a flexible endoscope having a rod that can be deformed via two rotary knobs. The control unit comprises a user interface comprising a first interface mounted on a pivotal support attached to a housing of the control unit, the first interface being engageable by the palm of a hand. The control unit further comprises a drive unit operable via the user interface, the drive unit comprising a first drive mechanism for engaging the two rotary knobs, thereby allowing a user to control the deflection of the endoscope shaft via the first interface.

### SUMMARY

The present invention aims to avoid the disadvantages of the prior art in two embodiments, either in the form of a separate module forming an accessory allowing upgrading of a commercial endoscope handgrip, or in the form of an endoscope handgrip natively having such a control module. In both embodiments, the invention consists of a mounting plate capable of being attached to the ulnar-palmar grasping zone of a handgrip of an endoscope, said mounting plate having an ulnar-palmar bearing surface extended by a heel extending in a direction forming an angle of 90°±25° with respect to the perpendicular to the plane of said ulnar-palmar bearing surface, said heel comprising an electromechanical sensor that delivers a control signal controlling the movement of an endoscopic instrument.

Within the meaning of this patent, the term “mounting plate” means a thin blade, with a thickness of less than 2 millimeters, flat or deformed to allow the rear face to fit the surface of the ulnar-palmar grasping zone of a handgrip of an endoscope. The term “ulnar-palmar grasping zone of a handgrip of an endoscope” is understood to mean the semi-tubular lateral surface of the Clean Specification handgrip extending between the two transverse front ends of the handgrip. The palm of the hand surrounds the handgrip, which is generally tubular in shape, the thumb extending on one side and the fingers (index, middle, ring and possibly little finger) fitting the ulnar-palmar grasping zone on the other side.

The invention relates more particularly to a motorized actuation module for an endoscopic instrument, characterized in that it consists of a mounting plate capable of being attached to the ulnar-palmar grasping zone of a handgrip of an endoscope, said mounting plate having an ulnar-palmar bearing surface extended by a heel extending in a direction forming an angle of 90°±25° relative to the perpendicular to the plane of said ulnar-palmar bearing surface, said heel comprising an electromechanical sensor delivering a control signal for controlling the movement of an endoscopic instrument.

Advantageously, said median ulnar-palmar grasping surface is extended on the opposite side by a motorized drive block, comprising a motorized mechanism of a filiform element for connection with said instrument, the lower end of which opens into said flexible rod to ensure connection with said distal instrument, said mounting plate further having means for connection with the handgrip of a flexible endoscope. Advantageously, said means for connecting with the handgrip of a flexible endoscope consist of an endpiece capable of being inserted into the working channel of an endoscopic rod. According to a preferred variant, the mounting plate comprises a motorized drive block comprising a motor placed below the endpiece with the inlet of the working channel of an associated endoscope. Advantageously, said heel is formed by a protrusion with a thickness of less than 3 millimeters having, at its end, a sensor whose actuating surface is defined by a generatrix forming an angle of angle of 90°±20° with the longitudinal axis of said handgrip.

According to a particular embodiment, the drive block, comprising a motorized mechanism of a filiform element for connection with said instrument, the lower end of which opens into said flexible rod of the endoscope to provide a connection with said distal instrument. According to a variant, said drive unit has a side actuating button controlling the emergency stop of the movement of said instrument.

The invention also relates to a flexible endoscope comprising a handgrip having suction and wash control buttons located above an ulnar-palmar grasping zone of the user's fingers, said handgrip being extended by a flexible rod having a working channel for the passage of an instrument, the movement of which is controlled by an electrical interface, characterized in that said interface has an ulnar-palmar bearing surface extended by a heel extending in a direction forming an angle of 90°±25° relative to the perpendicular to the plane of said ulnar-palmar bearing surface, said heel comprising an electromechanical sensor delivering a control signal controlling the movement of an endoscopic instrument.

Advantageously, said heel is formed by a protrusion having thickness of less than 3 millimeters having, at its end, a sensor whose actuating surface is defined by a generatrix forming an angle of angle of 90°±20° with the longitudinal axis of said handgrip. Advantageously, said sensor is a rotary sensor actuated by a wheel whose axis forms an angle between 0° and ±70° with respect to the longitudinal axis parallel to the longitudinal axis of said handgrip. Advantageously, the mounting plate further comprises, in the lower part of said ulnar-palmar grasping zone, a drive block comprising a motorized mechanism of a filiform element for connection with said instrument, the lower end of which opens into said flexible rod of the endoscope to provide a connection with said distal instrument.

Preferably, said drive block forms a protrusion relative to the surface of the lower part of the ulnar-palmar grasping zone, on the opposite side, relative to said ulnar-palmar grasping zone, at said extension constituting the actuation control. Advantageously, said control interface (100) further comprises a length selector for moving the instrument by a predetermined length. Preferably, said control interface further comprises means for coupling a peripheral of said instrument.

According to a variant, the endoscope comprises a motorized actuation module grouping together said interface and said drive block in the form of a detachable subassembly of the handgrip. According to another variant, said sensor has an interaction surface opposite said ulnar-palmar grasping zone of less than 50 mm².

### BRIEF DESCRIPTION OF THE DRAWINGS

The invention is described below, by way of non-limiting example, with reference to the appended drawings, in which:

FIG. 1 is an exploded partial perspective view showing an endoscope handgrip according to the prior art;

FIG. 2 is a ¾ front view of an endoscope handgrip according to the invention;

FIG. 3 shows a ¾ front view of an endoscope handgrip according to the invention;

FIG. 4 is a ¾ front view of a second endoscope handgrip variant according to the invention;

FIG. 5 shows a ¾ front view of a first variant of a control module according to the invention;

FIG. 6 shows a ¾ front view of a second variant of a control module according to the invention;

FIG. 7 is a ¾ front view of a third endoscope handgrip variant according to the invention;

FIG. 8 is a ¾ front view of a third endoscope handgrip variant according to the invention;

FIG. 9 is a ¾ front view of a second variant of a module according to the invention;

FIG. 10 is a view of the internal mechanism of this second variant of a module according to the invention;

FIG. 11 is a longitudinal sectional view of this second variant of a module according to the invention;

FIG. 12 is a bottom view of this second variant of a module according to the invention;

FIG. 13 is a perspective view of the connecting endpiece with the working channel of the endoscope;

FIG. 14 is a perspective view of a first configuration of a mounting plate according to the invention;

FIG. 15 is a perspective view of a second configuration of a mounting plate according to the invention;

FIG. 16 is a perspective view of a third configuration of a mounting plate according to the invention;

FIG. 17 is a perspective view of a fourth configuration of a mounting plate;

FIG. 18 is a perspective view of a fifth configuration of a mounting plate according to the invention; and

FIG. 19 is a perspective view of a sixth configuration of a mounting plate according to the invention.

### DETAILED DESCRIPTION

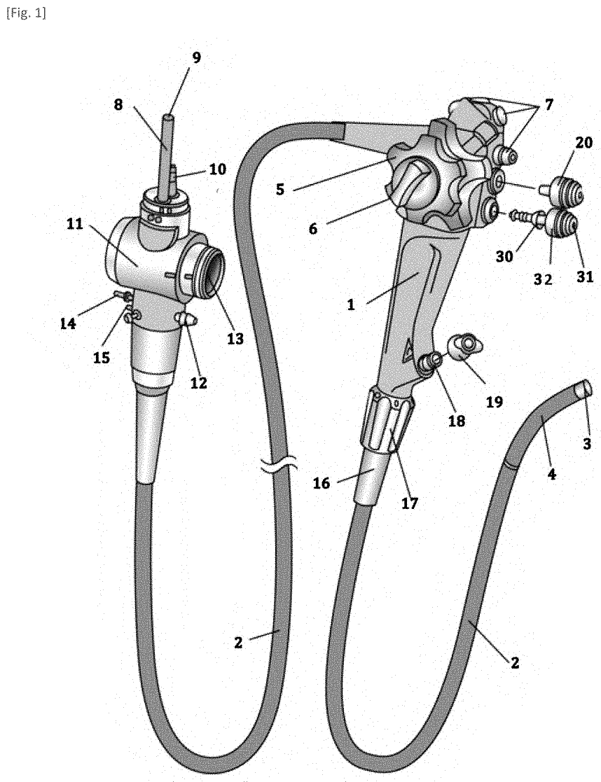

FIG. 1 shows an example of a known endoscope, which comprises a handgrip (1) from which extends a flexible tube (2) ending in a distal or operational end (3) that is introduced into the cavity to be explored and whose orientation can be controlled by a bendable sleeve (4). This sleeve is controlled by wires that are pulled in a controlled manner following the manual actuation of Clean Specification knobs or buttons (5, 6, 7) provided on the handgrip (1) of the endoscope. The lever control section allows the physician to control all functions of the endoscope. The bending (spatial orientation) levers (5, 6, 7) direct the bending cables and control the bending section at the distal end of the insertion tube, thereby enabling two-dimensional orientation. Locking mechanisms (brakes) allow the bendable section to be fixed in the desired position.

The light is transmitted by an optical fiber (8) whose proximal end (9) is coupled to an optical connector. The connection block (11) further comprises an air intake (10).

A pneumatic connector (12) is intended to be connected to a vacuum pump or a reversible air pump to control insufflation or aspiration at the distal end (3). The technical block (11) also comprises an electrical connector (13) and a connection support (14) for a safety cord and a water supply (15).

The handgrip (1) is connected to the tube (2) by a sheath (16) extended by a sleeve (17). The handgrip has a working channel opening (18) fitted with a disposable valve (19).

The handgrip comprises a piston (20) controlling the wash channel and a piston (30) controlling the aspiration or insufflation via two tubes extending between the handgrip (1) and the distal end (3), one to bring air and water to said distal end, the other to perform a biopsy or an aspiration. The application of suction is regulated by means of a piston (30) provided on the handgrip. The piston is joined to a neighboring duct by welded connections. The pistons (20, 30) are coupled to the body of the handgrip by annular bases (32).

The piston (30) connects the suction channel to the working channel in the insertion tube. By pressing the piston button (31), aspiration of the working channel can be performed. The air/water piston (20) is similar to the piston and to the suction piston (30), except that a piston with a two-way button is used in a dual-channel device that allows air or water to be conveyed to the lens at the distal end, for washing or blowing air to improve vision. The two pistons (20, 30) can be detached for replacement, when in use, or otherwise for cleaning.

### DETAILED DESCRIPTION OF THE CONTROL INTERFACE

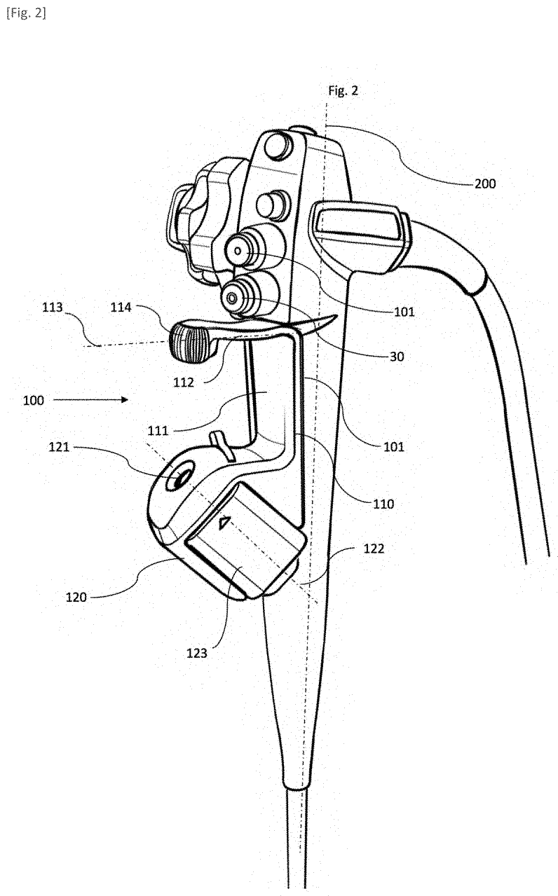

The handgrip according to the invention shown in FIG. 2 and following differs from “standard” handgrips by an additional function of motorized control of an endoscopic instrument introduced into the endoscopic tube (2) in order to perform a procedure at the distal end such as a sample, an electro-coagulation, a suture, an incision, etc. This motorized control is intended to move the endoscopic instrument forward or backward in the endoscopic tube (2), without an operator needing to push or pull the instrument. This motorized control does not relate to the possible actuation of the end of the endoscopic instrument, for example the opening or closing of a clamp provided at the distal end of the endoscopic instrument.

The aim is to allow the operator holding the handgrip (1) with one hand to continue to apply the usual controls, but also to control the movement of the endoscopic instrument to its working position without manual intervention, without requiring the use of the other hand, which is generally used to guide the endoscopic tube (2) in the introduction area, and without relying on the assistance of a second operator, which requires complex coordination. The movement of the instrument consists in progressing in the endoscopic tube along a trajectory corresponding to the median of the tube, forwards and backwards, to allow the active part of the instrument to reach the intervention zone. This is a movement relative to the endoscopic tube (2).

To do this, the handgrip (1) is equipped with a control interface (100) that is positioned on the handgrip (2) at the ulnar-palmar grasping zone (101) just below the piston (20) controlling the wash channel and the aspiration or insufflation control piston (30). This zone (101) is generally flat to allow support for the pads of at least two or three fingers, the index, middle, ring and little fingers.

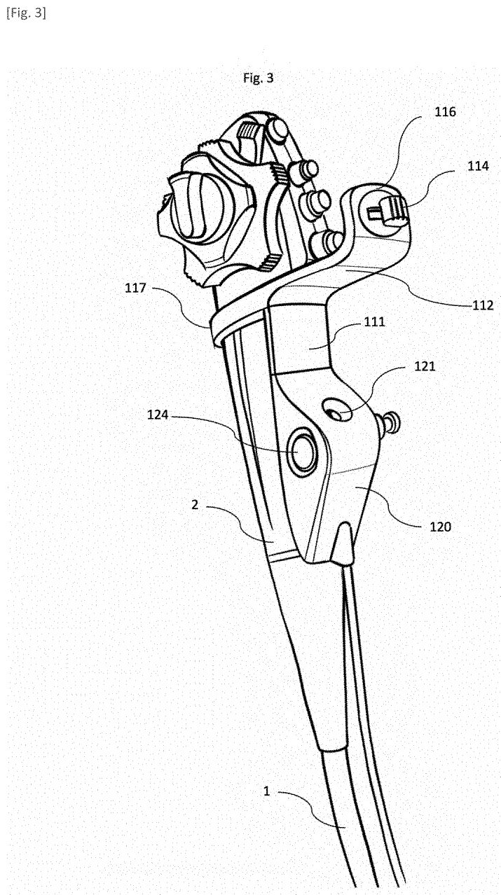

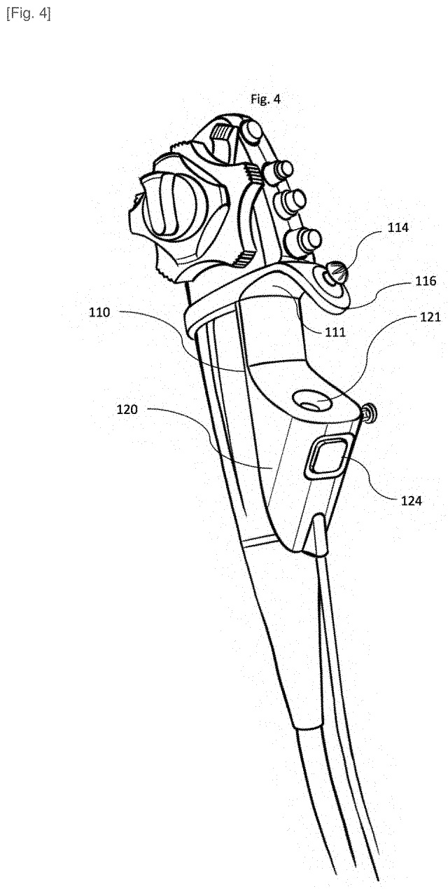

The module illustrated in FIGS. 2 and 3 consists of a thin mounting plate (110), approximately 1 millimeter, having a median ulnar-palmar support portion (111) that is superimposed on the ulnar-palmar grasping zone (101) of the handgrip (2), extending laterally over the handgrip. This median portion (111) is extended on one side by an extension (112) extending in a substantially transverse plane (113). Within the meaning of this patent, “substantially transverse plane” means a surface extending in a direction between +45° and −45° relative to the longitudinal axis (200) of the handgrip (2).

This extension (112) has a decreasing width, from an initial width corresponding to the width of the median zone (111), to an end of lesser width bearing the sensor provided with an actuating surface (114). In the above example, the sensor is a rotary electromechanical sensor with an actuating surface (114) formed by a toothed wheel in the form of a spherical zone allowing actuation with the pad of a finger, for example the middle finger or the ring finger, leaving the index finger available for the other usual handgrip commands. The other end of the mounting plate (100) is formed by a motorized block (120) whose longitudinal axis (122) is inclined with respect to the longitudinal axis (200) of the handgrip (2), with an angle comprised between ±30°(tilted up or down) and 90°(orientation perpendicular to the longitudinal axis (200) of the handgrip (2)).

This motorized block (120) has a median channel opening on each side of the block (120) through a port (121) for the passage of the translation element of the endoscopic instrument, to ensure its movement inside of the endoscopic tube (1) and its distal positioning for a procedure with this instrument. To this end, the block (120) comprises an electric motor or an electromagnetic actuator controlled by the sensor (114) via an electronic control circuit. It is powered by a rechargeable electric battery (123) engaged in a side connector. On the opposite wall, the block (120) has an emergency stop button (124).

In the embodiment illustrated by FIG. 2, the extension (112) is curved upwards at its end (116), which bears the sensor (114). Furthermore, the mounting plate is provided with a collar (117) for attachment around a standard handgrip (2). In the variant illustrated by FIG. 3, the emergency stop button (124) is arranged on the front face of the motorized unit (120), and the mounting plate (110) has an extension (111) extending transversely, with one end (116) angled upward to bear a sensor (114) operable by a hemispherical button.

### Embodiment in the Form of an Accessory Module

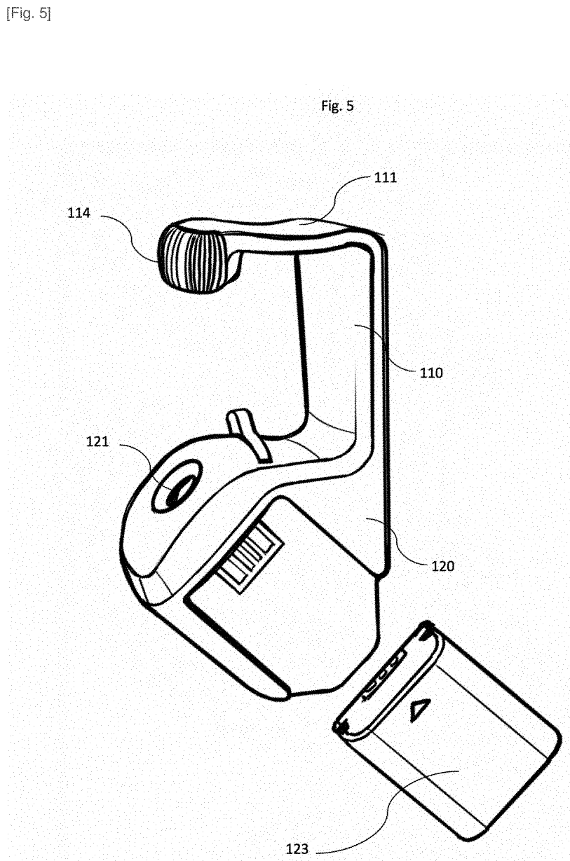

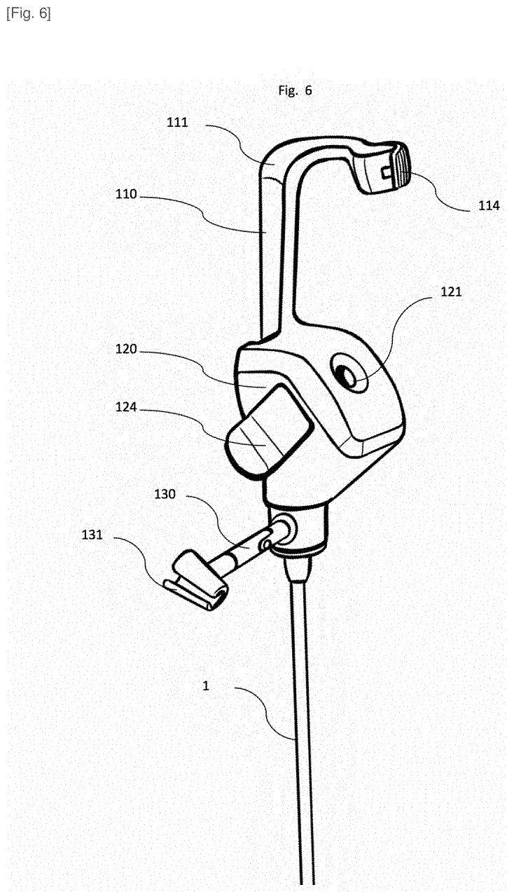

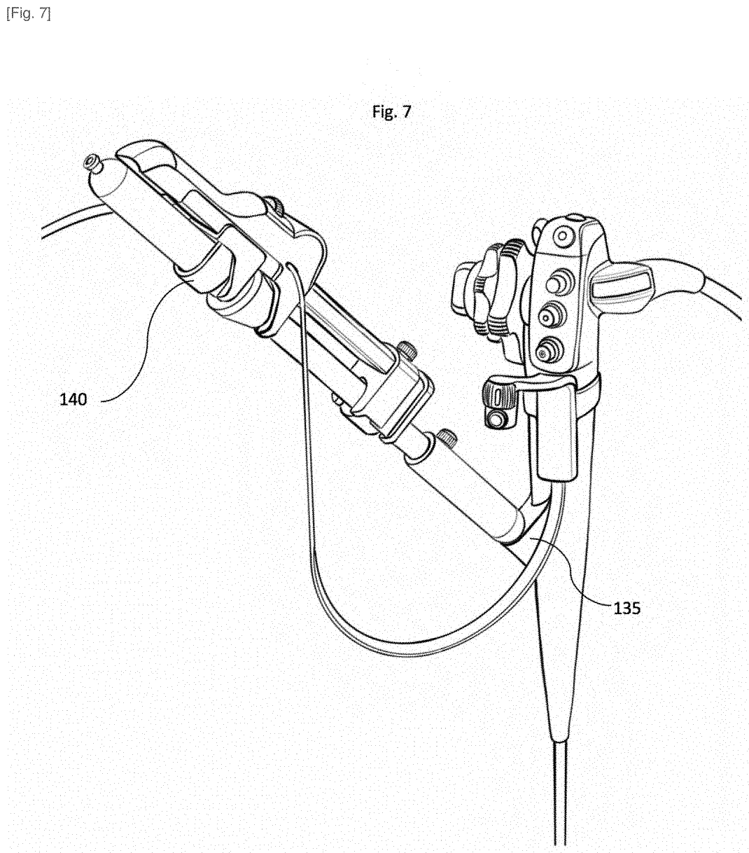

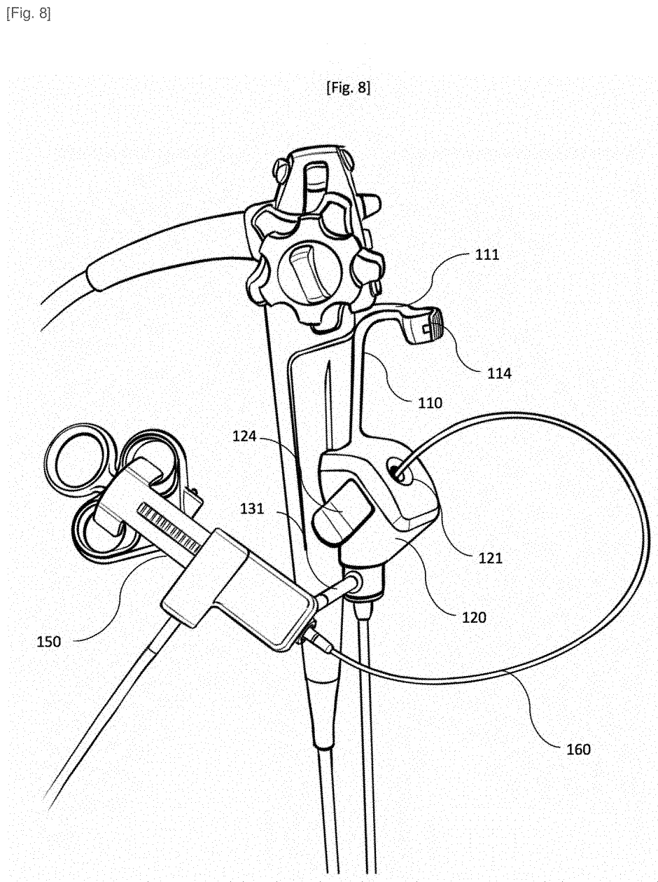

The interface can be integrated into a handgrip (2) or be produced in the form of an accessory that can be mounted and attached on a pre-existing handgrip (2). FIGS. 5 and 6 illustrate views of such an accessory, having the same technical characteristics as those previously described. The motorized block (120) is extended laterally by an adapter to receive a peripheral of the endoscopic instrument, for example an automated puncture equipment item (140) as shown in FIG. 7 or a mechanism (150) for controlling scissors or a biopsy needle as shown in FIG. 8. This adapter is constituted by an articulated arm (130) provided with an accessory support (131) or by a mechanical connector (135).

### Alternative Embodiment of an Accessory Module

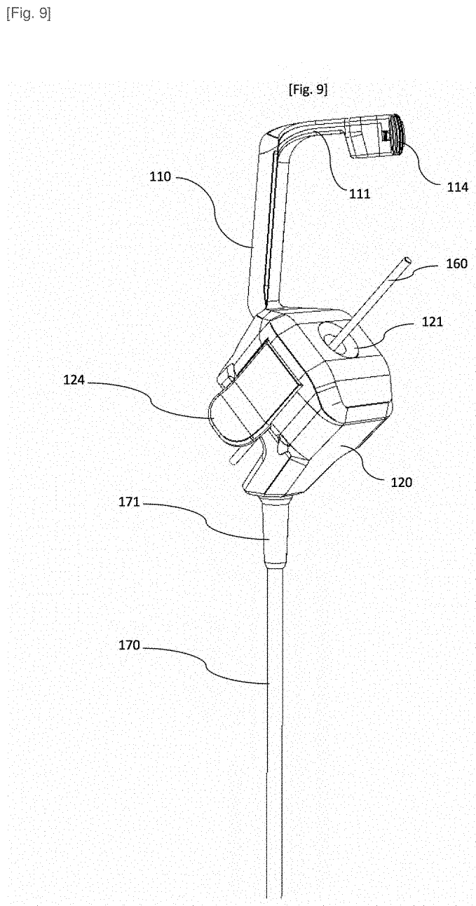

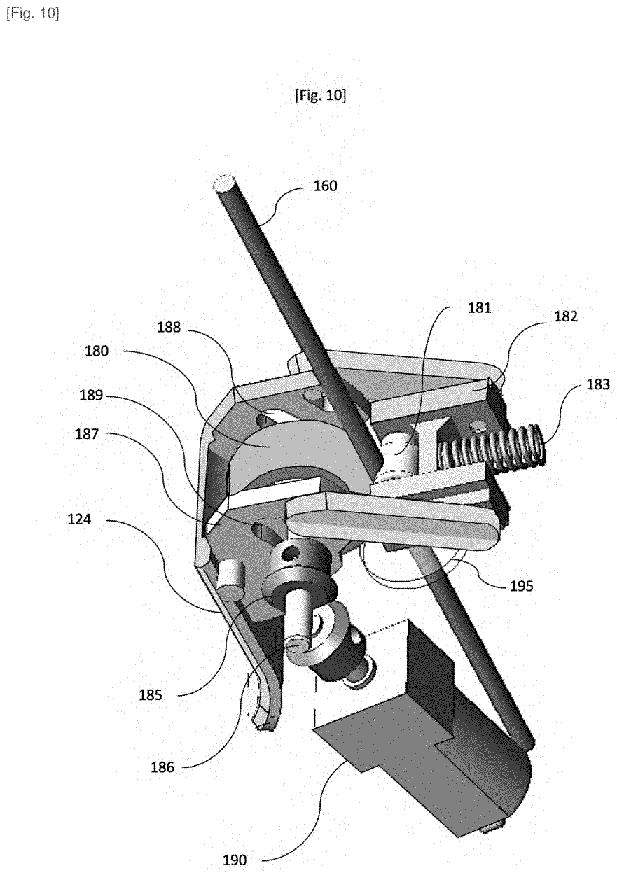

FIGS. 9 and 10 show an alternative embodiment of an autonomous module, constituting an accessory intended to equip a pre-existing endoscopic handgrip. The technical characteristics previously described are present in this variant. The motorized block (120) incorporates a mechanism for driving the filiform element (160) by a drive roller (180) coming into contact with the surface of this filiform element (160), a pressure roller (181) coming into diametrically opposite contact to ensure good adhesion. This pressure roller (181) rotates freely about an axis supported by a mobile carriage (182) pushed back in the direction of the roller (180) by a spring (183) oriented perpendicular to the axis of introduction of the filiform element (160). It is powered by an electric wire (170) protected by a sleeve (171).

The drive roller (180) is actuated by a motor (190) via a bevel gear (185). This motor (180) is controlled via the user control (114). The motor (190) is arranged in a substantially longitudinal direction, relative to the scrolling axis of the filiform element (160), with an angle of between 0° and ±30° relative to this axis to optimize the space requirement and weight distribution.

The motor (190) is positioned in the motorized block (120) below the interface (195), coming to be placed on the inlet of the working channel of the endoscope, in order to favor good balancing of the handgrip and to avoid raising the center of gravity of the handgrip fitted with this module. The axis (186) is guided by two arcuate slots (188, 189) formed on a frame (187) secured to the actuating button (124). In the intermediate position of the actuating button (124), the drive roller (180) ensures the movement of the filiform element (160), which is pressed against it by the free roller (181) with a controlled force.

When the actuating button (124) is pushed in the direction of the scrolling axis of the filiform element (160), in the direction of the handgrip when the module is mounted on the handgrip, it causes the uncoupling of the gears of the angle lever (185) together with the pressing of the drive roller (180) against the free roller (181), which blocks the scrolling of the filiform element (160). When, on the contrary, the actuating button (124) is moved in the opposite direction, by acting on its curved part with a finger slipped between the handgrip and the inner surface of the actuating button (124), the drive roller (180) is moved away from the free roller (181), and the filiform element (160) is thus released and can then be moved manually.

The operation of this actuating button (124) is very intuitive. It controls the instantaneous opening or closing of the tool engagement mechanism:
instantaneous engagement/disengagement of the tool moved by the filiform element (160);
safety by immediate stopping for advance/withdrawal of the tool moved by the filiform element (160);
instant switching between manual and automatic operation.

Manual operation of the tool allows a return to normal use of the endoscope without having to remove the motorized module.

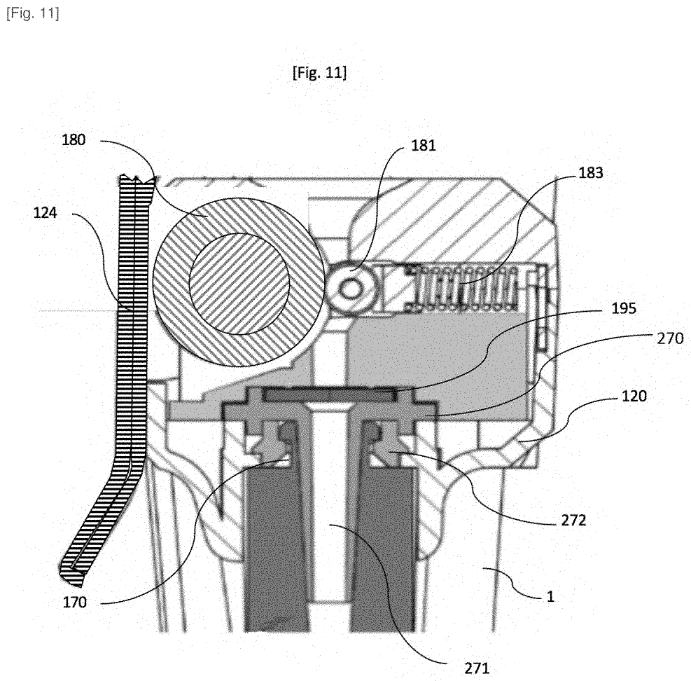

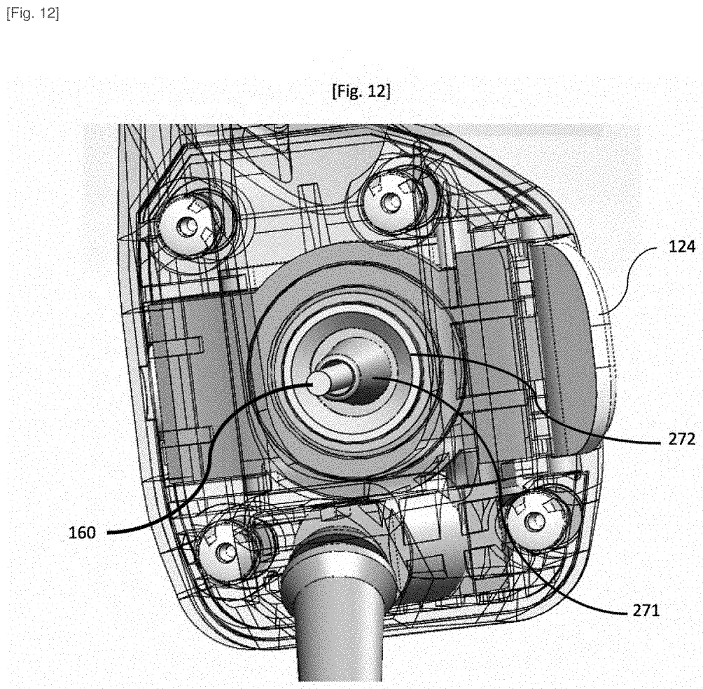

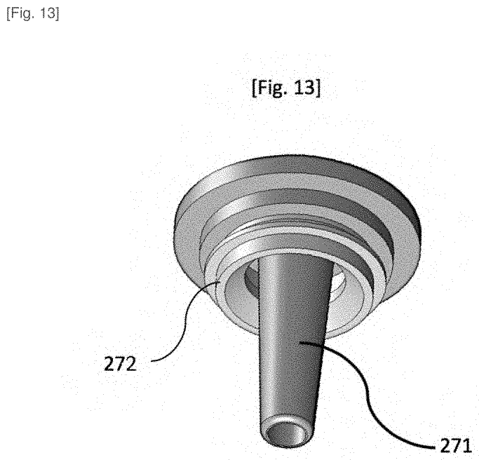

Attaching the instrument to the working channel of the endoscope (FIG. 11-13)

Attaching the module on the handgrip does not require an attachment element such as a collar or a clip, but is achieved by a frustoconical part shown in FIG. (13), having a flange (270) ensuring the seal between the working channel and the motorized module, extended by a tapered endpiece (271). Introducing into the working channel a “standard” endoscope of the longitudinal endpiece (271) that extends the distal end of the motorized block (120) ensures both the guidance of the filiform element (160) and the wedging of the module relative to the endoscopic handgrip. The generally tubular or tapered ring (271) has a seal (272) that seals the sheath (170) relative to the endoscopic guide. This seal (272) comprises a bead that engages by deformation and wedging around the end of the working channel (also comprising a bead).

The frustoconical endpiece (271) has, at its proximal end, a disc expansion (270) that engages in a complementary receiving zone provided on the front face of the motorized unit (120). The proximal end of the endpiece (271) has a circular space in which a seal (195) engages. This solution makes it possible to physically separate the working channel from the guide (1), from the motor and from the drive mechanism constituting the upper part of the motorized block (120). One or more fluid seals seal the fluid in the rod (1) passage.

Applications

The invention has multiple applications, which relate in particular to echo-endoscopically guided sampling with a fine needle (EUS-FNA), with cytological and histological study. Endoscopic ultrasound (EUS) has become an essential protocol for determining the stage of cancer in the gastrointestinal system, especially when combined with fine needle aspiration (FNA) or a fine needle biopsy (FNB) of body tissues. The FNA biopsy is performed using a dedicated needle called the FNA needle. During the procedure, an EUS imager is brought into contact with a body wall behind which the biopsy site is located, and then an FNA needle is advanced through the working channel of the EUS endoscope. The needle is advanced through the body wall to the site to be biopsied, usually a suspicious lesion, and negative pressure is applied to the inner end of the needle to retrieve tissue samples. The needle is then removed from the EUS field and the tissue sample is retrieved and analyzed.

The FNA needles available on the market are quite similar: they are intended to be engaged at the proximal end of the working channel (1), usually via the Luer lock, and comprise a hollow needle housed in a sheath. The FNA needle proximal end handle comprises:
a sheath adjuster used to bring the end of the sheath into contact with the body wall to be penetrated (with the needle unexposed). Once the correct sheath position is achieved, the sheath adjuster can be secured to the handgrip body;
a needle slider for sliding and exposing the needle past the distal end of the sheath to reach the biopsy site;
a safety ring that can be attached to the body of the handgrip to limit the longitudinal travel of the needle slider so that multiple needle insertions can be easily performed by the user;
a port at the proximal end of the handle for applying negative pressure to the hollow inner bore of the needle.

An FNA biopsy typically requires the physician to manually pass the biopsy needle multiple times, back and forth, at different locations within the target lesion. The action of passing the biopsy needle is not standardized, as the penetration depth and the passage speed are controlled by doctors. Usually, a long pass generally receives more sample than shorter passes, and a slow pass generally receives more sample than faster passes. Non-standardized sampling could result in suboptimal yield or non-reproducible biopsy sampling. The conventional method also produces a poor estimate of sample yield, which can lead to undersampling and diagnostic failure. This wastes time, likely delays patient treatment, and is associated with additional medical costs that could be avoided with a more standardized sampling method. A similar technical problem has been mentioned in patent DE10128336, a method has been proposed. This method of collecting cell samples by fine needle aspiration (FNA) biopsy consists in introducing a fine needle of a sampling device into the tissue, this fine needle being subjected to a vacuum; it is set in motion relative to the tissue, then the vacuum is released and the fine needle is withdrawn from the tissue.

The present invention provides a device with a tool control interface that improves control of biopsy needle sampling, standardizing tissue sampling that allows better reproducibility, planning and estimation of tissue samples. With prior art handgrips, actuation of the needle slider requires the endoscopist to leave his right hand on the axis of the endoscopic sight, which results in a loss of control of the endoscope and decreased accuracy. This is an important limitation because reaching different locations in the lesion is critical for diagnostic yield. A system that allows the user to operate an FNA needle while keeping one hand on the handgrip and the other on the rod of the endoscope is therefore desirable.

The control interface according to the invention can advantageously be used to control an FNA needle, the user keeping his right hand on the rod of the endoscope. The movement of the shaft and the needle can be controlled by operating the control interface.

Clean Specification

In a preferred embodiment, needle insertion is controlled by the right hand using the control interface, and a one-dimensional drive control allows for slow and precise needle operation. Once the needle is inserted to the desired depth, a second Boolean sampling control (on/off) can be used to repeat the motion: once the sampling control is pressed, the needle is retracted and reinserted at the same distance. This feature allows the user to make multiple passes with a similar depth while varying the position of the scope (ventilation technique) to reach multiple sites within the lesion. The needle can then be withdrawn using the drive controller and it can be withdrawn from the endoscope.

According to a variant, the control interface (100) further comprises a length selector to allow the user to directly define the penetration depth of the needle. When the user presses the sample control, the needle is inserted to the depth set by the length selector and returned to a default position.

In one embodiment, the control interface (100) is used in conjunction with an FNA needle integrating at least one linear actuator adapted to translate the needle. The actuator is connected to the control interface physically or wirelessly. The FNA needle is also connected to a power source, either via a physical connection with the control interface or by integrating a battery. In another embodiment, the control interface is connected to an “EUS device” adapted to be mounted on an available FNA needle and connected to the control interface.

Summary of the “biopsy” application
1) An endoscope according to the invention, or a standard endoscope supplemented by a module according to the invention, comprises two parts intended for the operation of the endoscopic instrument: a user control interface and a drive unit.
2) The user control interface is mounted on the handgrip (2) of the endoscope and just below the suction and irrigation buttons (20, 30). The user control (114) comprises a first control button for applying a relatively slow movement command, and optionally a sampling button that continuously advances and retracts the biopsy needle for a fixed number of passes at the predefined length, set by the length selector.
3) Before attaching the drive unit to the sight, the needle safety ring should be tightened securely, ideally to the maximum length. This allows the needle to travel the maximum distance and to have the center weight point closer to the scope handgrip.
4) The drive unit consists of a linear actuator consisting of two parts, a non-sliding part and a sliding part. Each part containing an attachment means that is mounted on the biopsy needle device. The attachment means on the non-sliding part is attached to the safety ring of the needle device. The attachment means on the sliding part is attached to the sliding handgrip of the biopsy device.
5) The drive also comprises a lever-operated safety mechanism. When it is disabled, manual control of the needle device is possible.
6) To take a biopsy sample, the endoscopist advances the needle near the desired location using the drive controller or manually. A desired length is set on the length selector on the sliding part of the drive unit. A needle is advanced into the lesion via the drive control button. The desired needle penetration length can be adjusted, if necessary, until the needle arrives at the desired location in the target.
7) Once the needle is in the target and the desired penetration length is selected, multiple passes of the needle can be made. When the user presses the sample button, the linear actuator housed in the drive unit is activated. The linear actuator brings the connectors closer to each other for a preset time. This movement of the linear actuator causes the needle to advance and retract to the preset distance based on the length selector.
8) When a set of passes is complete, the user can manually reposition the needle and readjust the desired penetration length, if necessary. Steps 8 and 9 are repeated until enough samples are collected.
9) Documentation of the sampling procedure—the user records each preselected length and the corresponding number of passes made by pressing the sampling button.

“Forceps” Application

Common endoscopic tools, such as forceps and collets (for example), require manual opening or closing of the tool handgrip (see FIG. 8). The tool handgrip is usually controlled by an assistant under the supervision of an endoscopist, and not directly by an endoscopist. Communication, however, may be difficult, as the exact degree of closing/opening and the movement to be performed may be difficult to verbalize. This usually requires an experienced assistant or a long learning curve between an assistant and the endoscopist to achieve more effective communication. With good communication, it is still difficult to avoid miscommunication, which may lead to operation errors, extra operation time and safety issues. One solution is to provide automatic control when closing and opening the tool via a user control interface, so that endoscopists can control their own tool.
1) The solution proposed by the present invention applies perfectly to the control of an endoscopic instrument requiring an opening or closing control and which is attached to a tool holder adjacent to the handgrip of the endoscope.
2) It consists of a connector that extends from the bottom of the drive unit and a tool holder that connects to the connector.
3) The tool holder consists of a linear actuator with two parts: a sliding part and a non-sliding part. At least a portion of the endoscopic tool is mounted on the tool holder.
4) Open and closed endoscopic tools usually consist of a sliding and a non-sliding (non-moving) part. A sliding part of the device is mounted on the sliding part of the tool, while the non-sliding part of the device is mounted on the non-sliding part of the tool.
5) The opening or closing of the tool is controlled by the position/movement of the sliding part, which can be controlled by the user via a user control interface (similar to the original control) or foot pedals.

Modulation of the Movement Speed of the Endoscopic Instrument

Some endoscopic procedures require different speed controls, including slow speed for precise motion control and rapid advancement that allows perforation through the membrane of a structure. According to a variant, the invention makes it possible to provide two different speed control buttons on the user control interface, which allow more valuable control of the speed of the device, or even an indexed sensor making it possible to select the appropriate speed using the control button (114).

Module Setup

The configuration of the heel (112) of the actuation module can assume different forms, depending on the nature of the sensor that it bears. By way of non-limiting examples, six particular configurations are presented below.

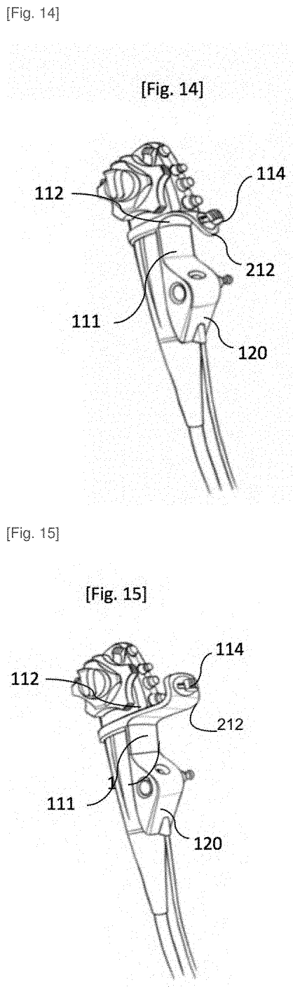

FIG. 14 is a perspective view of a first configuration of a mounting plate according to the invention. The heel (112) extends in a direction substantially perpendicular to the bearing surface (111), then has a curved area, in the direction of the motorized drive block (120) to form a substantially parallel (or slightly inclined with an angle of the order of 30°) surface (212) bearing a sensor provided with a button that can be actuated laterally, from right to left, and pressed, for example to control the advancement or the retreat of a tool and the blockage thereof.

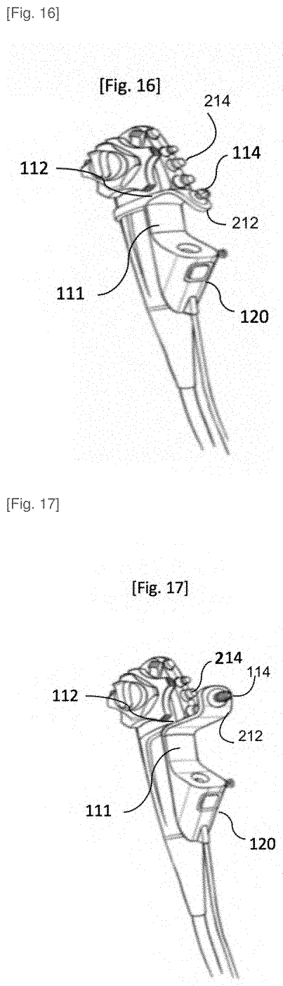

FIG. 15 is a perspective view of a second configuration of a mounting plate according to the invention similar to the previous configuration, except that the bending is in the direction opposite the motorized block. FIG. 16 is a perspective view of a third configuration of a mounting plate according to the invention where the bending is in the same direction as in the first configuration, the surface (212) bearing a wheel operable in different directions, of the “joystick” type, with possible actuation by pushing. FIG. 17 is a perspective view of a fourth configuration of a mounting plate according to the invention where a second actuator (214) is mounted on a curved plate (212) connected to the engine block (120).

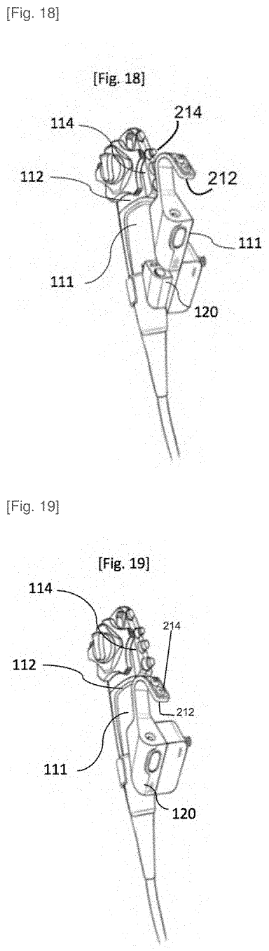

FIG. 18 is a perspective view of a fifth configuration of a mounting plate according to the invention with a second curved plate (212) bearing several sensors or buttons (214) superimposed on the first sensor (114), the two sensors (114) and (214) being in staggered planes. FIG. 19 is a perspective view of a sixth configuration of a mounting plate according to the invention with a second curved plate (212) bearing several sensors or buttons (214) staggered longitudinally relative to the first sensor (114), the two sensors (114) and (214) being in staggered planes.

1. A motorized actuation module for an endoscopic instrument, comprising a mounting plate configured to be attached to an ulnar-palmar grasping zone of a handgrip of an endoscope, said mounting plate having an ulnar-palmar bearing surface extended by a heel extending in a direction forming an angle of 90°±25° relative to a perpendicular to a plane of said ulnar-palmar bearing surface, said heel comprising an electromechanical sensor delivering a control signal operable to control movement of said endoscopic instrument.

2. The motorized actuation module according to claim 1, wherein said median of said ulnar-palmar bearing surface is extended on an opposite side by a motorized drive block, comprising a motorized mechanism of a filiform element for connection with said instrument, a lower end of which opens into a flexible rod of said endoscope to ensure connection with said instrument, said mounting plate further being connected with said handgrip of said endoscope which is flexible.

3. The motorized actuation module according to claim 2, comprising a connection of said handgrip of said endoscope comprises an endpiece configured to be inserted into a working channel of said flexible rod of said endoscope.

4. The motorized actuation module according to claim 2, wherein said motorized drive block comprises a motor placed below said endpiece.

5. The motorized actuation module according to claim 1, wherein said heel is formed by a protrusion having thickness of less than 3 millimeters having, at its end, and a sensor whose actuating surface is defined by a generatrix forming an angle of 90°±20° with the longitudinal axis of said handgrip.

6. The motorized actuation module according to claim 1, wherein it further comprises, in a lower part of said ulnar-palmar grasping zone, a drive block comprising a motorized mechanism of a filiform element for connection with said instrument, a lower end of which opens into a flexible rod of said endoscope to provide a connection with said instrument

7. The motorized actuation module according to claim 6, wherein said drive block has a side actuating button controlling an emergency stop of movement of said instrument.

8. A flexible endoscope comprising a handgrip having suction and wash control buttons located above an ulnar-palmar grasping zone of a user's fingers, said handgrip being extended by a flexible rod having a working channel configured to allow passage of an instrument, movement of which is controlled by an electrical interface, said interface including an ulnar-palmar bearing surface extended by a heel extending in a direction forming an angle of 90°±25° relative to a perpendicular to a plane of said ulnar-palmar bearing surface, said heel comprising an electromechanical sensor delivering a control signal controlling the movement of an endoscopic instrument, another end of said interface (100) being formed by a drive block.

9. The flexible endoscope according to claim 8, wherein said heel is formed by a protrusion having thickness of less than 3 millimeters having, at its end, and a sensor whose actuating surface is defined by a generatrix forming an angle of 90°±20° with a longitudinal axis of said handgrip.

10. The flexible endoscope according to claim 9, wherein said sensor is a rotary sensor actuated by a wheel whose axis forms an angle between 0° and ±70° with respect to a longitudinal axis parallel to said longitudinal axis of said handgrip.

11. The flexible endoscope according to claim 8, wherein it further comprises, in a lower part of said ulnar-palmar grasping zone, a drive block comprising a motorized mechanism of a filiform element connected with said instrument, a lower end of which opens into said flexible rod of said endoscope to provide a connection with said instrument.

12. The flexible endoscope according to claim 8, wherein said drive block forms a protrusion relative to a surface of a lower part of said ulnar-palmar grasping zone, on an opposite side, relative to said ulnar-palmar grasping zone, at said extension constituting an actuation control of said movement of said instrument.

13. The flexible endoscope according to t claim 12, wherein said drive block has a side actuating button controlling an emergency stop of said movement of said instrument.

14. The flexible endoscope according to claim 8, wherein said control interface further comprises a length selector for moving said instrument by a predetermined length.

15. The flexible endoscope according to claim 8, wherein said control interface further comprises a coupling configured to connect to a peripheral of said instrument.

16. The flexible endoscope according to claim 8, wherein it comprises a motorized actuation module grouping together said interface and said drive block in a detachable subassembly of said handgrip.

17. The flexible endoscope according to claim 8, that wherein said sensor has an interaction surface opposite said ulnar-palmar grasping zone of less than 50 mm².
